(19) United States
(12) Patent Application Publication
(10) Pub. No.: US 2016/0199553 A1
(43) Pub. Date: Jul. 14, 2016

(54) BREAST PUMP ADAPTER

(71) Applicant: Luft Industrie Inc., Oakland, CA (US)

(72) Inventor: Oliver Albers, Oakland, CA (US)

(21) Appl. No.: 14/991,445

(22) Filed: Jan. 8, 2016

Related U.S. Application Data
(60) Provisional application No. 62/101,710, filed on Jan. 9, 2015.

(51) Int. Cl. A61M 1/06

(52) U.S. Cl. CPC A61M 1/066

(57) ABSTRACT

Breast pump adapter, breast pump system, and adapter kit are provided, as well as a method of using same. The adapter allows coupling of a breast shield assembly with a storage container (e.g., milk bottle), with an airtight, sealed fluid path. Compatibility of a bottle coupling of the breast shield assembly with a milk bottle, for example, is not necessary with the adapter. The breast pump adapter can include an elongated body having a first end and a second end and an intermediate central region. At least one of the first and second ends can be formed of a flexible material configured to stretch around a breast shield assembly bottle coupling or around a storage container opening to form an airtight seal. A housing can optionally be included as part of a breast pump adapter kit.

### RELATED APPLICATIONS

This application claims the benefit of U.S. Provisional Patent Application No. 62/101,710, filed Jan. 9, 2015, the content of which is incorporated herein by reference in its entirety.

### FIELD OF INTEREST

The present inventive concepts relate to the field of breast pumps, and in particular, to an apparatus and method for transferring breast milk to a storage device.

### BACKGROUND

Breast pumps of various kinds are used to facilitate the expression and storage of breast milk for mothers who nurse.

There are two general categories of breast pumps: electric pump and manual hand pump. The primary difference between the two categories is the apparatus used to create suction for promoting the expression of breast milk. Beyond that, the breast pump includes several components that interface with the pump, regardless of whether electric or manual, and forms a sealed fluid path from the breast to a milk bottle. These components can be referred to as a shield assembly.

The shield assembly includes a shield or breast cup that receives a breast and, when suction is applied, creates a seal with the breast to maintain the force of the suction. The shield connects to a neck that includes a pump valve. And at the opposite end of the neck, distal from the shield, is a bottle coupling. The bottle coupling is internally threaded and mates with a corresponding bottle, which are different for different breast pumps.

When in use, a sealed fluid path exists from the shield to the bottle, with suction applied by a pump via the valve. Expressed milk flows into the bottle and once filled the bottle can be capped and stored for later use.

Different breast pumps use different sized bottles. Because of the size and threading of the bottle coupling, only certain bottles can be used with certain shield assemblies. Existing adapter kits provide a selection of different sized parts for mixing and matching to find a proper combination that will work between a specific bottle and a specific shield assembly of the breast pump.

### BRIEF SUMMARY

In accordance with aspects of the inventive concept, provided is a breast pump adapter that enables coupling of a breast shield assembly with a storage container (e.g., milk bottle), with breast pump adapter maintaining a sealed fluid path between the breast shield assembly and the storage container. Compatibility of a rigid threaded bottle coupling of the breast shield assembly with a milk bottle, for example, is not necessary with the adapter. In its preferred form, the adapter is a single-piece universal adapter that fits a variety of bottles and a variety of breast pump shield assemblies, without the need for mixing and matching different parts to couple to the bottle to the shield assembly. The adapter can include flexible portion, so that one end stretches over and around and seals a bottle opening and the other end stretches over and around and seals a bottle coupling of a breast shield assembly. An intermediate structure or feature of the adapter can provide increased rigidity to the adapter for gripping, increased resistance against collapsing during use in response to suction of the breast pump, or both. The increased rigidity can be achieved by an increased thickness, density, or both provided by the intermediate structure. Here, “intermediate” means anywhere between flexible coupling ends of the adapter.

In accordance with one aspect of the inventive concept, provided is a breast pump adapter comprising an elongated body having a first end and a second end and an intermediate region. At least one of the first and second ends is formed of a flexible material configured to stretch around a breast shield assembly bottle coupling or around a storage container opening to form an airtight seal.

In various embodiments, the first end is formed of the flexible material and configured to stretch around a storage container opening to form an airtight seal. In such embodiments, or other embodiments, the second end may optionally be threaded to mate with threads of the breast shield assembly bottle coupling.

In various embodiments, the second end is formed of the flexible material and configured to stretch around a breast shield assembly bottle coupling. In such embodiments, or other embodiments, the first end may optionally be threaded to mate with threads of the storage container opening.

In various embodiments, only the first end is formed of the flexible material and configured to stretch around a breast shield assembly bottle coupling. In such embodiments, the second end can be a rigid piece configured to couple to a specific bottle.

In various embodiments, only the second end is formed of the flexible material and configured to stretch around a storage container opening to form an airtight seal. In such embodiments, the first end can be a rigid piece configured to couple to a specific breast shield assembly bottle coupling.

In various embodiments, only the first end can be formed of a flexible material configured to stretch around a breast shield assembly bottle coupling to form an airtight seal.

In various embodiments, only the second end can be formed of a flexible material configured to stretch around a storage container opening to form an airtight seal.

In various embodiments, the storage container can be a bottle or other breast milk storage device.

In various embodiments, the intermediate region can be formed of one material and at least one of the first and second ends can be formed of a different material.

In various embodiments, the adapter can be formed of a single piece of material.

In various embodiments, the adapter can be a single molded piece.

In various embodiments, the material can be or include at least one of silicone and/or rubber.

In various embodiments, at least one of the first end or the second end can be flared so that at least one of the first end and the second end has an opening that is wider than a width of an interior of the adapter at the intermediate region.

In various embodiments, the first and second ends are both flared.

In various embodiments, at least one of the first and second ends can include a pull tab that facilitates stretching of the corresponding first end and/or second end.

In various embodiments, the first end can be stretchable for coupling to and sealing the breast pump connector and the second end can be stretchable for coupling to and sealing the storage container.

In various embodiments, the intermediate region can include a substantially rigid region that resists collapsing under a vacuum pressure applied to via the breast shield assembly by a breast pump operatively coupled thereto.

In various embodiments, the substantially rigid region can be more rigid than the first and/or second ends of the adapter.

In various embodiments, the substantially rigid region can be thicker, denser, or both than the first and/or second ends of the adapter.

In various embodiments, the substantially rigid region can comprise a ring formed about a central region of the adapter.

In various embodiments, the breast pump adapter can further comprise a rigid housing configured to receive, surround, and secure therein a portion of the adapter.

In various embodiments, a length of the housing is less than a length of the adapter and the first end of the adapter extends beyond a first end of the housing and a second end of the adapter extends beyond a second end of the housing.

In various embodiments, wherein the adapter includes a protrusion in the intermediate region and the housing includes an intermediate channel configured to receive the corresponding protrusion of the adapter to secure the adapter within the housing.

In various embodiments, the protrusion includes the central ring and the channel is configured to receive and secure therein the central ring.

In various embodiments, the geometries of the first and second ends of the adapter can be asymmetric with respect to the intermediate region. In some embodiments, the first end can be longer than the second end, and vice versa.

In various embodiments, the breast pump adapter further includes a wall that extends from the first end to the second end, and wherein a portion of the wall at the intermediate region has a thickness that is greater than a thickness of portions of the wall at the first and second ends, respectively.

In accordance with another aspect of the inventive concept, provided is a breast pump system, comprising: a breast pump having a breast shield, a pump valve, and a bottle coupling; a breast pump adapter that can be coupled between the breast pump bottle coupling and a storage container opening to form an airtight fluid path between the breast pump bottle coupling and the storage container. The breast pump adapter comprises an elongated body having a first end and a second end and an intermediate region, wherein the first end is foimed to couple to the breast pump bottle opening and the second end is formed of a flexible material that is configured to stretch around a storage container opening to form an airtight seal therewith.

In various embodiments, the first end is also formed of the flexible material that is configured to stretch around and form an airtight seal with the breast pump bottle opening, and the intermediate region includes a substantially rigid region that resists collapsing under a vacuum pressure applied via the breast shield by a breast pump.

In accordance with another aspect of the inventive concept, provided is a breast pump adapter, comprising an elongated body having a first end, a second end, and an intermediate region. The first end is formed of a flexible material that is configured to stretch around a breast shield assembly bottle coupling to form an airtight seal. The second end is formed of a flexible material that is configured to stretch around a storage container opening to form an airtight seal. And the intermediate region includes a substantially rigid region that resists collapsing under a vacuum pressure applied via the breast shield assembly by a breast pump.

In accordance with another aspect of the inventive concept, provided is a method of coupling a breast pump to a storage container as described in reference to the figures, using a breast pump adapter as claimed.

In accordance with another aspect of the inventive concept, provided is a breast pump adapter as shown and described.

In accordance with another aspect of the inventive concept, provided is a breast pump adapter kit as shown and described.

### BRIEF DESCRIPTION OF THE DRAWINGS

The present invention will become more apparent in view of the attached drawings and accompanying detailed description. The embodiments depicted therein are provided by way of example, not by way of limitation, wherein like reference numerals refer to the same or similar elements. The drawings are not necessarily to scale, emphasis instead being placed upon illustrating aspects of the invention. In the drawings:

FIG. 1A is a front view of a breast pump adapter, in accordance with aspects of the inventive concept;

FIG. 1B is a side view of the breast pump adapter of FIG. 1A;

FIG. 1C is a cross-sectional view of the breast pump adapter of FIGS. 1A and 1B, taken along line C-C of FIG. 1B;

FIG. 1D is a top view of the breast pump adapter of FIGS. 1A-1C;

FIG. 2 is perspective of the breast pump adapter of FIGS. 1A-1E being attached to a bottle and a breast shield assembly;

FIG. 3 is a perspective view of the breast pump adapter of FIG. 2 attached to a bottle and a breast shield assembly;

FIG. 4A is perspective view of another embodiment of a breast pump adapter, in accordance with aspects of the inventive concept;

FIG. 4B is a side view of the breast pump adapter of FIG. 4A;

FIG. 4C is a cross-sectional view of the breast pump adapter of FIGS. 4A and 4B, taken along line D-D of FIG. 4B;

FIG. 4D is a top view of the breast pump adapter of FIGS. 4A-4C;

FIG. 5A is a perspective view of an embodiment of a breast pump adapter kit including the adapter of FIG. 1 and a housing in disassembled form, according to aspects of the inventive concept;

FIG. 5B is a perspective view of the breast pump adapter kit of FIG. 5A with the housing assembled;

FIG. 6A(1) is a side view of an embodiment of a pump-side piece of the housing of FIGS. 5A-B;

FIG. 6A(2) is a side view of a cross section of the pump-side piece of FIG. 6A(1) taken along line A-A of FIG. 6A(1);

FIG. 6A(3) is a bottom view of the pump-side piece of FIGS. 6A(1)-(2), with the bottom being the threaded end of the pump-side piece;

FIG. 6A(4) is a top view of the pump-side piece of FIGS. 6A(1)-(3), with the top being the flared end of the pump-side piece;

FIG. 6B(1) is a side view of an embodiment of a bottle-side piece of the housing of FIGS. 5A-B;

FIG. 6B(2) is a side view of a cross section of the bottle-side piece of FIG. 6B(1) taken along line B-B of FIG. 6B(1);

FIG. 6B(3) is a bottom view of the bottle-side piece of FIGS. 6B(1)-(2), with the bottom being the flared end of the bottle-side piece;

FIG. 6B(4) is a top view of the pump-side piece of FIGS. 6B(1)-(3), with the top being the flared end of the pump-side piece, with the top being the threaded end of the bottle-side piece;

FIG. 7A is a perspective view of the breast pump adapter kit of FIG. 5B;

FIG. 7B is a side view of the breast pump adapter kit of FIGS. 5B and 7A;

FIG. 7C is a cross-sectional side view of the breast pump adapter kit of FIG. 7B taken along line E-E of FIG. 7B;

FIG. 7D is a magnified view of an interface of the two threaded ends of the breast pump adapter housing and internally secured adapter of FIGS. 7A-7C;

FIG. 8 is an illustration of the breast pump adapter kit of FIGS. 5A-7D being attached to a bottle and breast shield assembly;

FIG. 9 is a perspective view of the breast pump adapter kit of FIG. 8 attached to a bottle and breast shield assembly;

FIG. 10 is a side view of an embodiment of an adapter having one end that is flexible and another end that is externally threaded;

FIG. 11 is a cross-sectional view of an embodiment of the adapter of FIG. 10 as a two-piece adapter;

FIG. 12 is a cross-sectional view of an embodiment of the adapter of FIG. 10 as a one-piece adapter;

FIG. 13 is a side view of an embodiment of an adapter having one end that is flexible and another end that is internally threaded;

FIG. 14 is a cross-sectional view of an embodiment of the adapter of FIG. 13 as a two-piece adapter;

FIG. 15 is a cross-sectional view of an embodiment of the adapter of FIG. 13 as a one-piece adapter;

FIG. 16A is a front view of another embodiment of a breast pump adapter, in accordance with aspects of the inventive concept;

FIG. 16B is a top view of the breast pump adapter of FIG. 16A;

FIG. 16C is a cross-sectional view of the breast pump adapter of FIG. 16A;

FIG. 16D is a perspective view of the breast pump adapter of FIG. 16A;

FIG. 16E is a perspective view of the breast pump adapter of FIG. 16A having a bottle end stretched for coupling to a bottle opening;

FIG. 16F is a perspective view of the breast pump adapter of FIG. 16E coupled to the bottle opening;

FIG. 16G is a perspective view of the breast pump adapter of FIG. 16A having a breast pump end stretched for coupling to a breast shield assembly;

FIG. 16H is a perspective view of the breast pump adapter of FIG. 16G coupled to the breast shield assembly; and

FIG. 16I is a perspective view of the breast pump adapter of FIGS. 16A, F, and H coupled to both a bottle and a breast shield assembly.

### DETAILED DESCRIPTION

Various exemplary embodiments will be described herein with reference to the accompanying drawings, in which some exemplary embodiments are shown. The present inventive concept may, however, be embodied in many different forms and should not be construed as limited to the exemplary embodiments set forth herein.

It will be understood that, although the terms first, second, etc. are used herein to describe various elements, these elements should not be limited by these terms. These terms are used to distinguish one element from another, but not to imply a required sequence of elements. For example, a first element can be termed a second element, and, similarly, a second element can be termed a first element, without departing from the scope of the present invention. As used herein, the term “and/or” includes any and all combinations of one or more of the associated listed items.

It will be understood that when an element is referred to as being “on” or “connected” or “coupled” to another element, it can be directly on or connected or coupled to the other element or intervening elements can be present. In contrast, when an element is referred to as being “directly on” or “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, steps, operations, elements, and/or components, but do not preclude the presence or addition of one or more other features, steps, operations, elements, components, and/or groups thereof.

Conventional breast pump assemblies include a bottle coupling that is generally constructed to include a threaded interface for coupling a breast shield assembly to a baby bottle. The threaded end of the bottle coupling is typically formed of a rigid plastic material, which may not be incompatible with many baby bottles, requiring a user to “mix and match” the shield assemblies with different baby bottles and pumps to arrive at a usable combination.

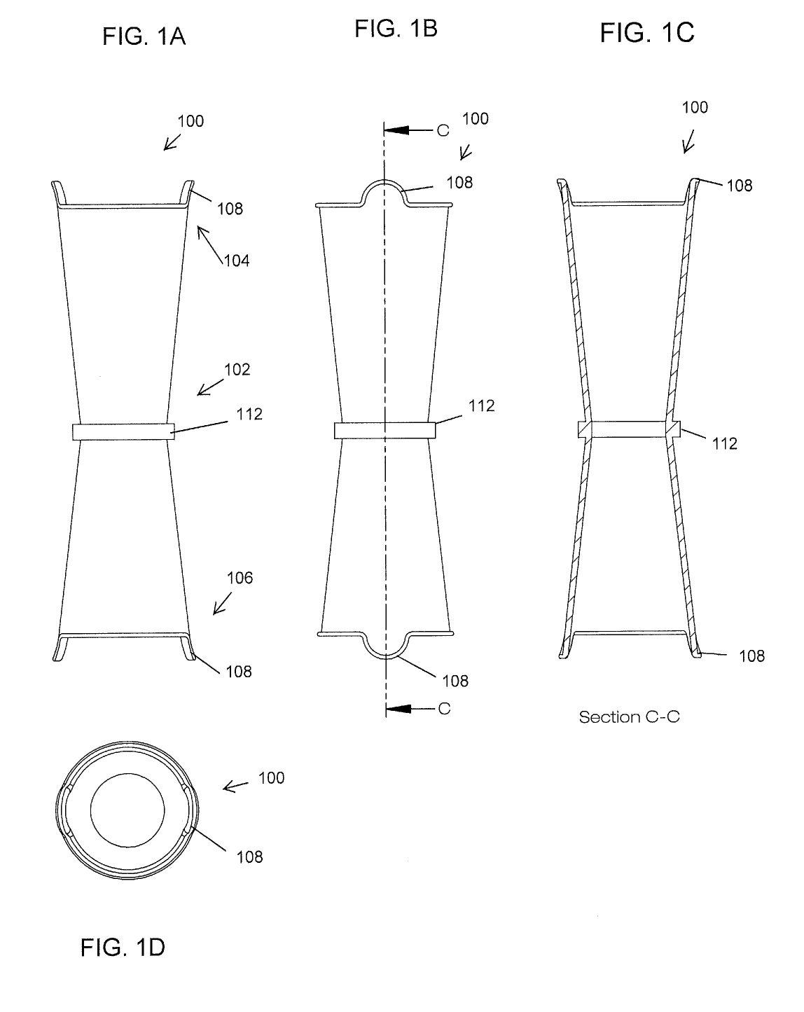

FIG. 1A is a front view of a breast pump adapter 100, in accordance with aspects of the inventive concept. FIG. 1B is a side view of the breast pump adapter 100 of FIG. 1A. FIG. 1C is a cross-sectional view of the breast pump adapter 100 of FIGS. 1A-1B, taken along line C-C of FIG. 1B. FIG. 1D is a top view of the breast pump adapter 100 of FIGS. 1A-1C.

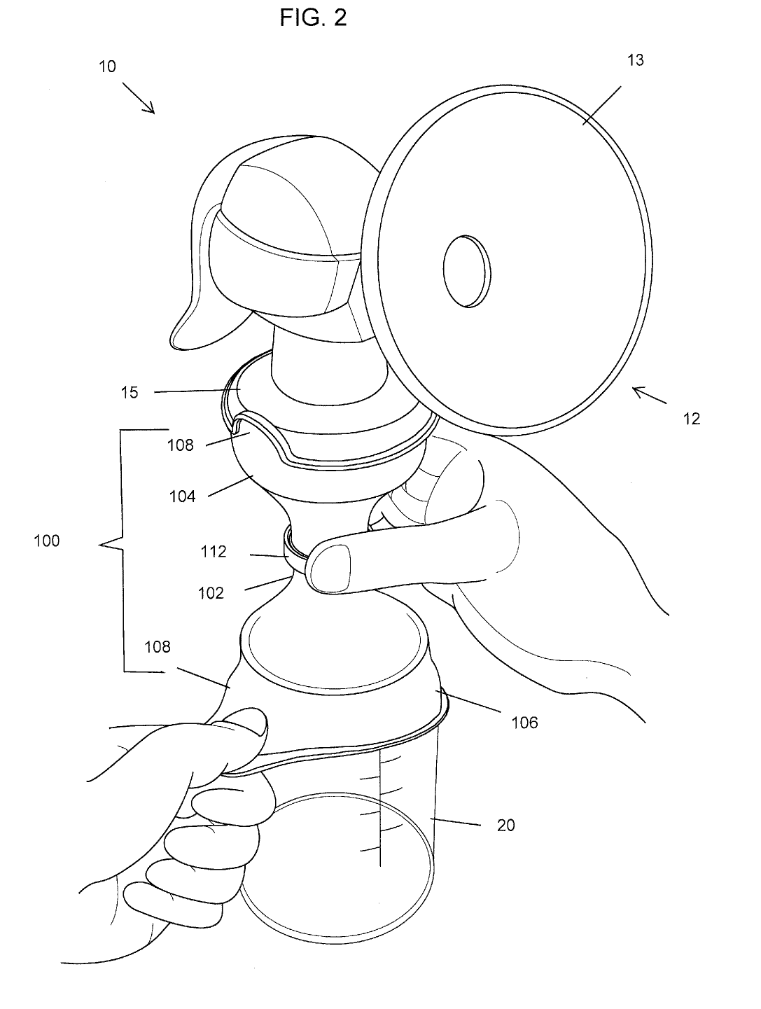

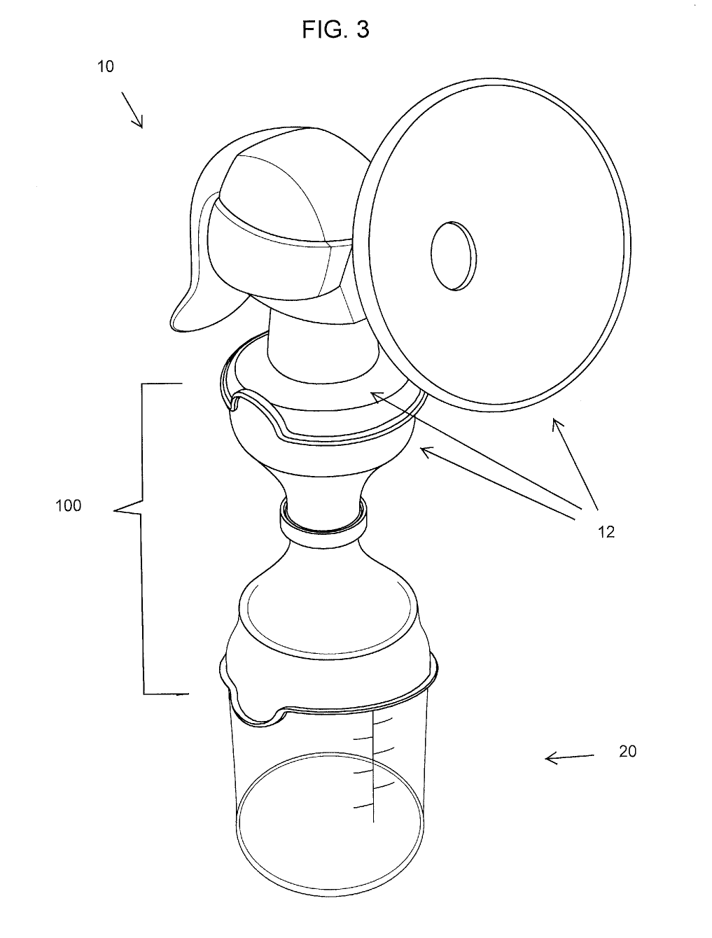

In this embodiment, the breast pump adapter 100 takes a hollow, generally tubular form. The adapter 100 is configured to create an enclosed, airtight fluid path between a shield assembly 12 of a breast pump 10 and a storage container 20, such as a bottle shown for example at FIG. 2. The adapter 100 includes a central region 102, a first end 104, and a second end 106. The portion of the adapter 100 between the first end 104 and second end 106 may be referred to as an intermediate region, inclusive of the central region 102. The first end 104 of the adapter 100 is constructed and arranged to receive and seal a bottle coupling 15 of a breast shield assembly 12 (see FIGS. 2 and 3), or other apparatus adapted to receive milk from a breast. The second end 106 of the adapter 100 is constructed and arranged to receive and seal an opening of a storage container 20, such as a milk or baby bottle.

Optionally, the first end 104 and second end 106 of the adapter 100 can each include one or more tabs 108, or lips or other gripping structures that, as shown in FIG. 2, can be held by a user to facilitate stretching the respective end when coupling the end to a shield assembly bottle coupling 15 or to a bottle opening, depending on the end.

The first end 104, the second end 106, and intermediate region can be formed of the same material, or different materials. In some embodiments, the entire adapter 100 can be flexible, or alternatively, the intermediate region or central region 102 can be rigid while the first 104 and second 106 ends are flexible. In yet other embodiments, only one of the first end 104 and second end 106 can be flexible. By “flexible” it is meant that an end is sufficiently elastic (or stretchable) and durable so that it can be stretched around and seal a bottle opening or shield assembly bottle coupling 15 without tearing or breaking. Flexible materials used for the first end 104 and/or second end 106 can be one or more non-toxic, sufficiently elastic materials, such as silicone, rubber, or the like. To be sufficiently elastic, the material, when coupled to a bottle coupling 15 or bottle opening, preferably tightly conforms to an external surface that is substantially smooth, threaded, ridged, textured, or rough, such as would be typically found with breast pumps and bottles.

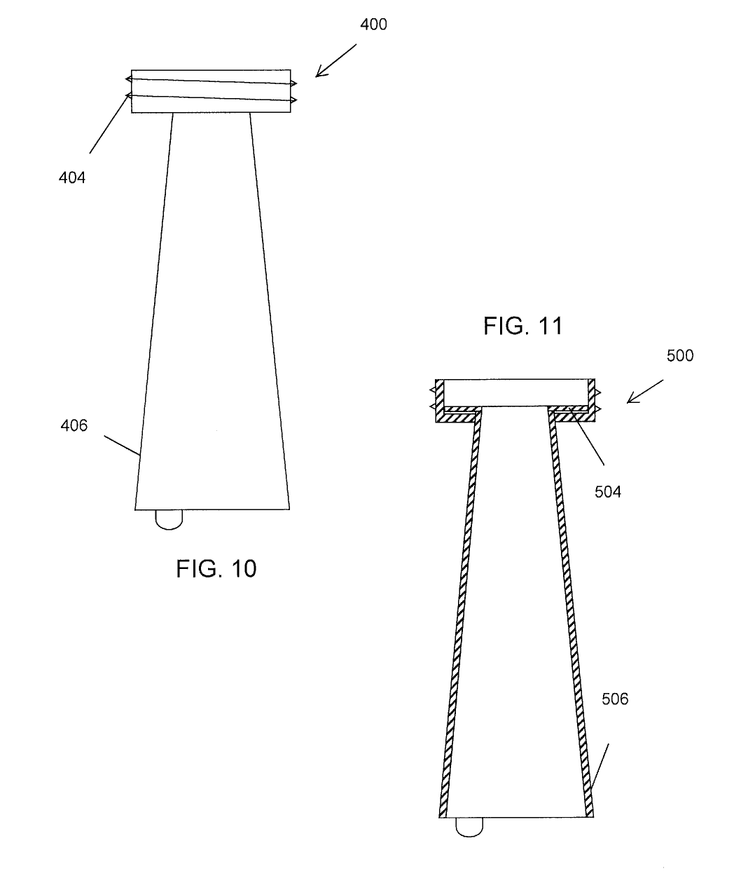

The seal formed by at least one of the first end 104 or the second end 106 of the adapter 100 is a conformal air tight seal that maintains the suction or vacuum of a breast pump, whether electric or manual. The depicted embodiments show two flexible (adaptable) ends. But in other embodiments, only one end is flexible and the other end can be rigid, e.g., one end can be internally threaded to mate with a bottle or externally threaded to mate with a bottle coupling of a shield assembly, while the other end is flexible to be adaptable to a wide variety of shield assemblies or bottle openings. See, as examples, FIGS. 10-15.

As described herein, the adapter 100 is constructed and arranged for positioning between a breast shield assembly 12 if a breast pump 10 and a storage container 20. For example, the dimensions or geometries of the adapter 100 provide for the forming of an airtight seal between the first end 104 and a surface of a bottle coupling of the pump 10 and between the second end 106 and a surface of an opening of a storage container 20. In doing so, the first end 104 has a width, diameter, or other geometry that is less than that of the bottle coupling 15 when the first end 104 is in a static, or unstretched, state. Similarly, the second end 106 of the adapter 100 has a width, diameter, or other geometry that is less than that of the bottle opening when the second end 106 is in a static, or unstretched, state. The present invention is not, however, inherently limited to the shape or configuration of the adapter 100 illustrated in FIGS. 1A-1D.

For example, the adapter 100 may have a tubular or cylindrical shape, instead or a funnel, flared or tapered shape.

Along these lines, in some embodiments, the central region 102 has an internal diameter, width, fluid path, cross-section or other such dimension that is smaller than that of the first end 104 and/or the second end 106. Accordingly, the adapter 100 can have or be formed in the shape of a first funnel or taper that extends and narrows from the first end 104 toward the central region 102 and/or a second funnel that extends and narrows from the second end 106 toward the central region 102. In some embodiments, one or more of the first 104 and second 106 ends can be flared. In such embodiments, therefore, the central region 102 may be narrower than one or both of the first end 104 and second end 106, or at least a fluid path within the central region 106 may be narrower than the first end 104 and/or second end 106. The first end flare can be adapted to couple to a bottle coupling 15 of a shield assembly 12 and the second end funnel can be adapted to couple to a bottle opening, wherein the adapter 100 forms an enclosed, airtight fluid path extending from the bottle coupling 12 to the bottle opening.

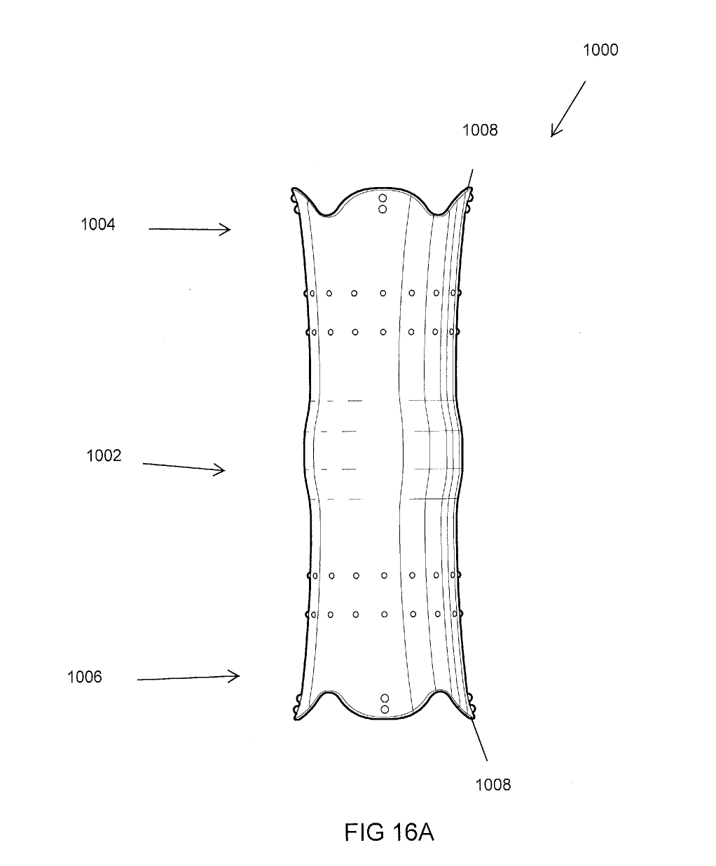

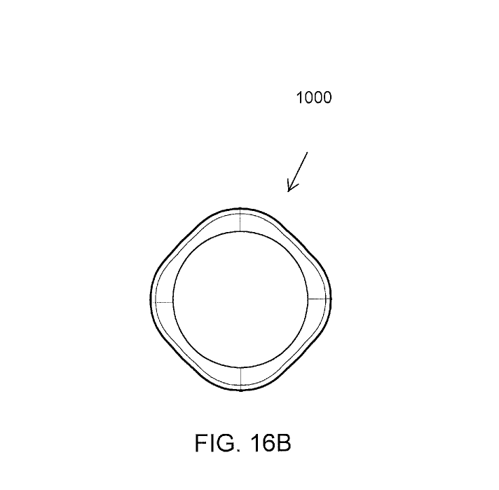

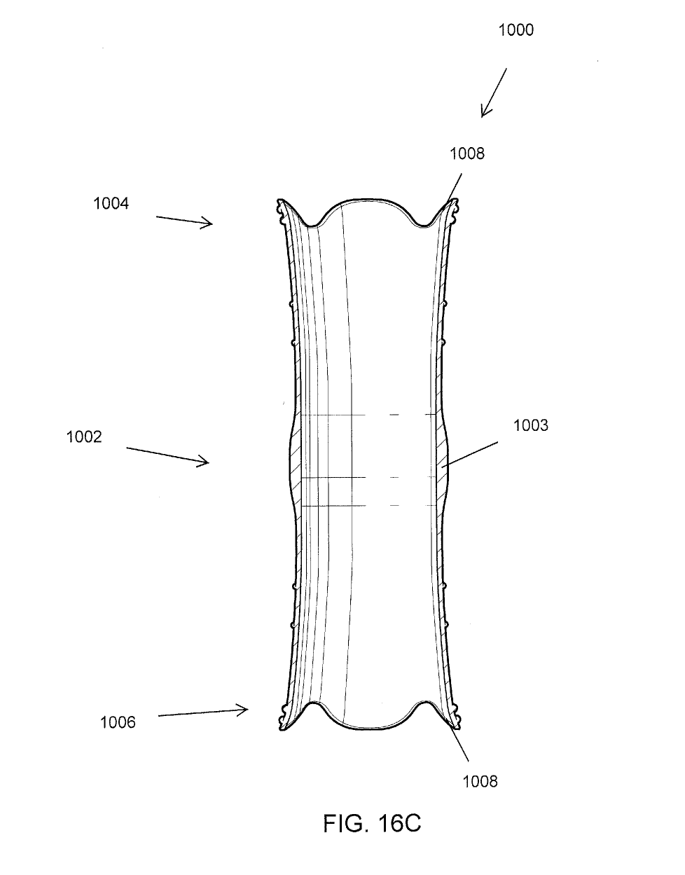

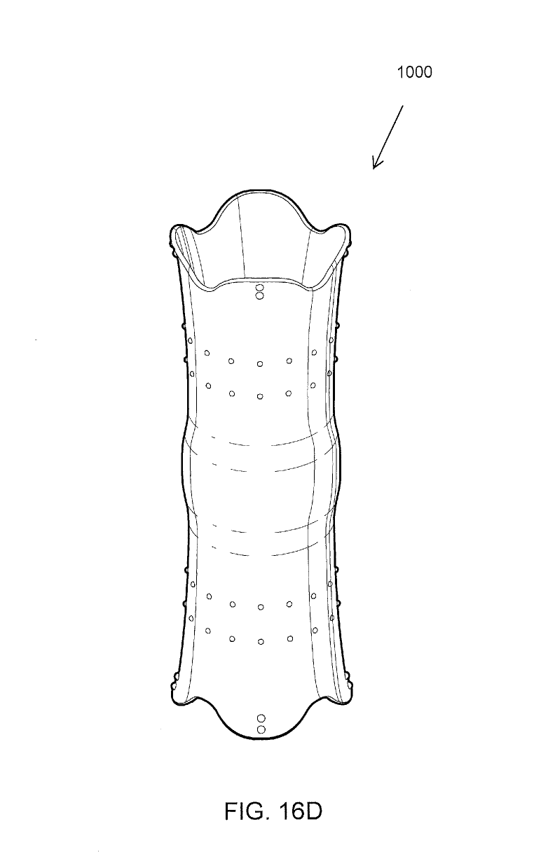

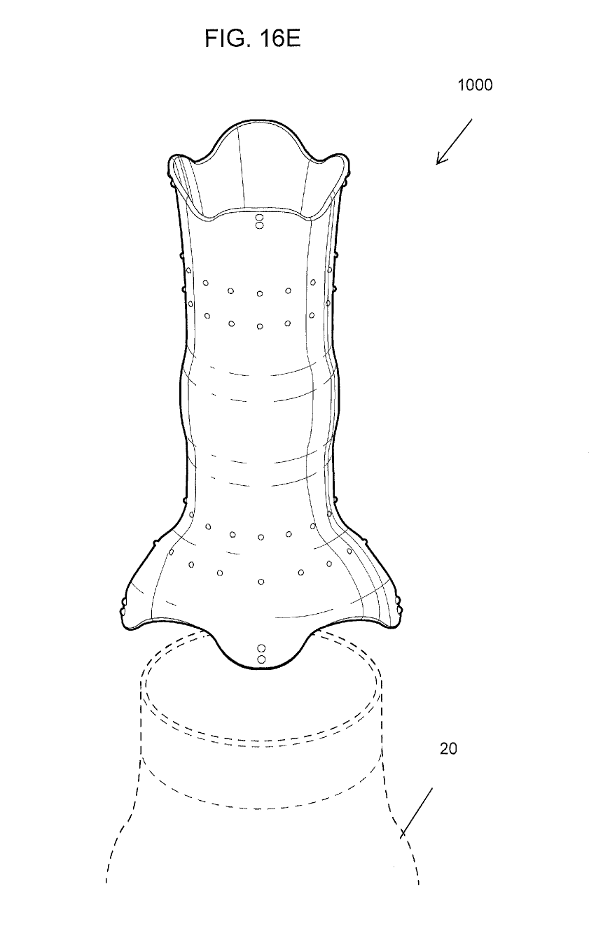

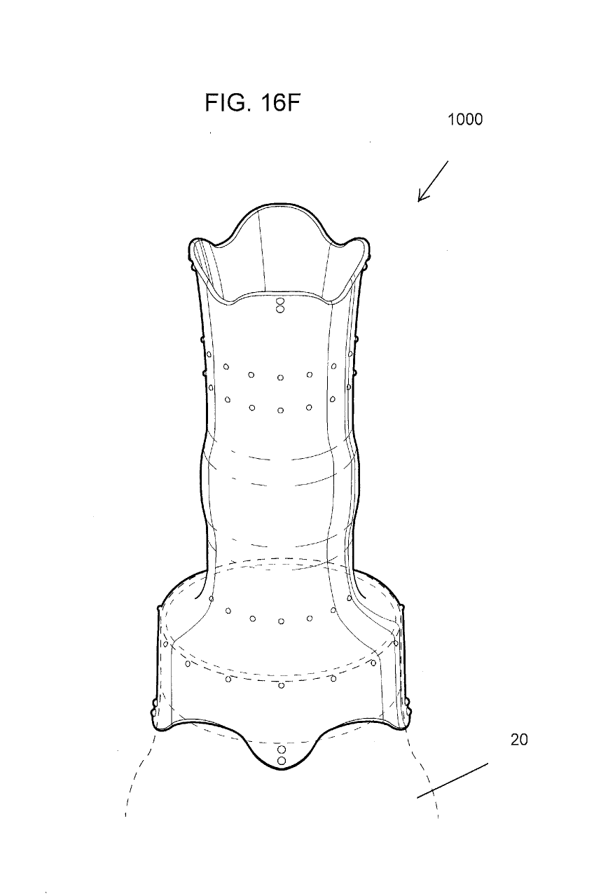

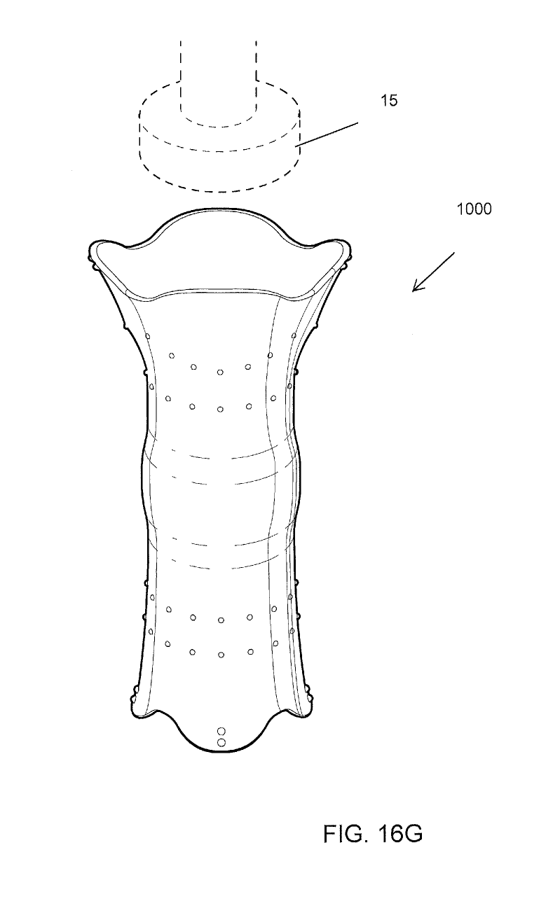

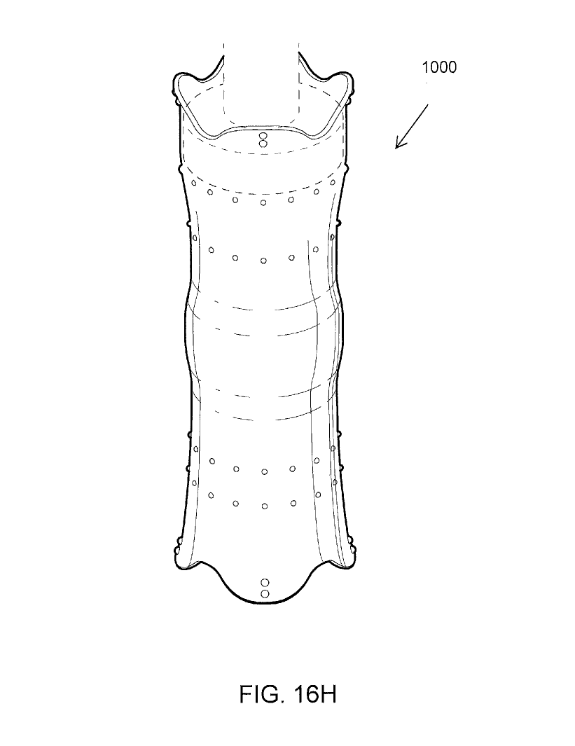

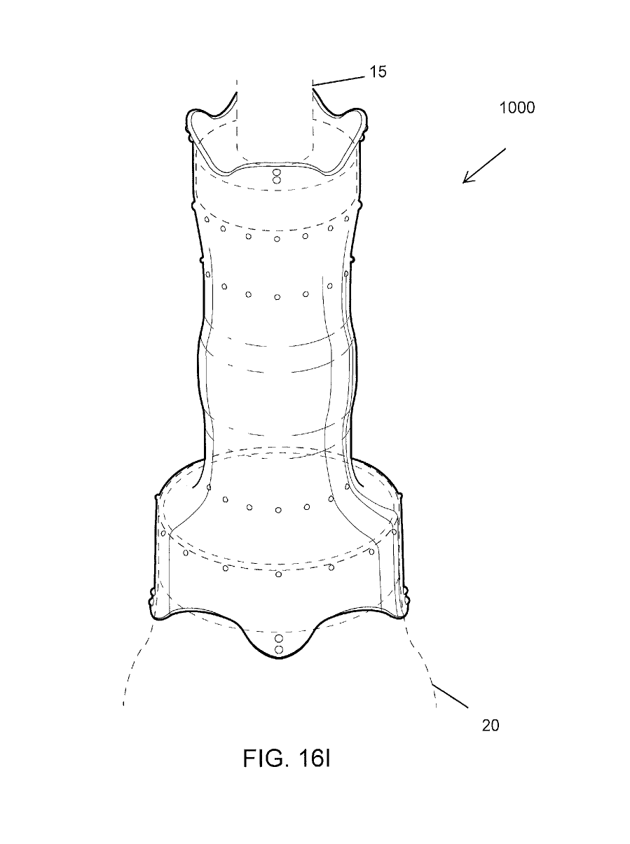

The adapter 100 may optionally include a support portion 112 in the central region. In various embodiments, the support portion 112 can provide: (1) stability and rigidity to the adapter; (2) a gripping area for holding the adapter; or (3) both. The support portion 112 can take various forms in various embodiments, including one or more projection or protrusion, one or more depressions or cavities, or combinations thereof. The support portion in some embodiments could include a thickening of the walls of the adapter in an intermediate (or substantially central) location of the adapter (for example, shown in FIGS. 16A-16I), which could be provided in combination with protrusions, projections, cavities, depression, or combinations thereof. The support portion 112 could have greater density than other portions of the adapter 100. In various embodiments, the support portion 112 is provided in the intermediate or central region 102 of the adapter 100 to provide sufficient stability and/or rigidity to prevent collapsing of the adapter 100 during use, e.g., when a vacuum is applied via the breast pump shield from the breast pump 10.

In various embodiments, the support portion 112 can include one or more outwardly protruding rings, as shown in FIGS. 1A-1C, which may be useful for providing stability and/or a gripping member in configurations where the adapter 100 is formed of a flexible material. And FIG. 2 shows the single ring 112 being used as a gripping element and as a mechanism to prevent collapsing of the narrowed central portion 102 of the adapter during use.

In some examples, the support portion 112, e.g., the single ring, can be formed of a different material than other portions of the adapter 100, i.e., different from the first end 104 and/or second end 106. Alternatively, the adapter 100 can be molded or machined from a single material to include the support portion 112, where the support portion 112 can be thicker and/or denser than the first and second ends 104, 106 of the adapter 100. In some embodiments, such as those discussed below, the support portion 112 can be formed to interface with a fluted housing or the like formed to surround and contain the sides or portions of the sides of the adapter 100, as described below.

The bottle coupling 15 of the breast shield assembly 12 is typically an internally threaded opening (not shown), i.e., threaded about an interior surface of the bottle coupling. An external surface of the bottle coupling 15 can be smooth, patterned, or textured. In use, the first end 104 of the adapter 100 is stretched about the exterior surface of the bottle coupling 15 to enclose and seal the opening with an airtight or fluid tight seal (see FIGS. 2 and 3). A tab 108 on the first end 104 of the adapter 100 is pulled to facilitate stretching of the first end 104 about the bottle coupling 15. FIGS. 2 and 3 show the first end in place for use.

The opening of the storage container 20, e.g., bottle, is typically an externally threaded opening (not shown), i.e., the thread is at an outer surface of the container. The threading of the bottle opening is formed to be compatible with the internal threading of a specific breast shield assembly bottle coupling 15. The second end of the adapter 106 is stretched about the externally threaded region of the bottle opening to enclose and seal the opening with an airtight or fluid tight seal. In use, a tab 108 on the second end of the adapter 100 is pulled to facilitate stretching of the second end 106 about the bottle opening. FIG. 2 shows the stretching and FIG. 3 shows the second end 106 as well as the first end 104 in place for use.

The first and second adapter ends 104, 106 may have asymmetric geometries, depending on the embodiment. For example, as shown in FIG. 1B, the first end 104 may be longer than the second end 106 or vice versa, when measured from a support member 112 located in the central region 106. Thus, in some embodiments, the distance from the support member 112 to the first end 104 and the distance from the support member 112 to the second end 106 may not be equal—one can be longer or shorter than the other. In other examples, the first and second ends 104, 106 may have symmetric geometries, e.g., length, width, surface area, and so on.

As described herein, in various embodiments the first end 104 and second end 106 each provide a sufficient airtight seal to permit a vacuum from the breast pump to be maintained when the adapter is used. For example, referring to FIGS. 2 and 3, a manual pump 10 is shown. The pump 10 can generate a vacuum at a breast shield 13 that causes the breast to express milk. The vacuum draws the milk through a valve in the breast shield assembly 12 and into the hollow interior of the adapter 100, entering via the first end 104 and exiting via the second end 106. The expressed milk, therefore, flows from the shield through the adapter to the storage container (e.g., milk bottle) coupled to the second end of the adapter. The adapter 100 is constructed and arranged so as not to impede or otherwise negatively affect the flow of milk from the breast pump 10 to the storage container 20.

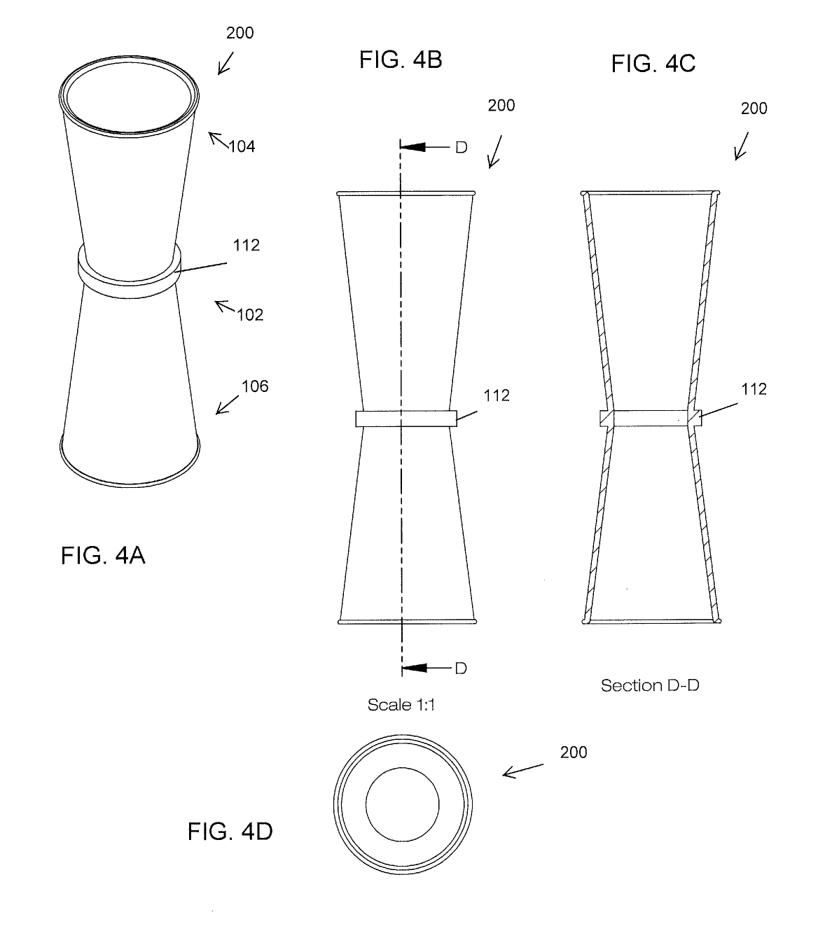

FIGS. 4A-D show another embodiment of an adapter 200, according to aspects of the inventive concept, having first and second ends 104, 106 and an intermediate region 102. FIG. 4A is perspective view of another embodiment of a breast pump adapter, in accordance with aspects of the inventive concept. FIG. 4B is a side view of the breast pump adapter of FIG. 4A. FIG. 4C is a cross-sectional view of the breast pump adapter of FIGS. 4A and 4B, taken along line D-D of FIG. 4B. And FIG. 4D is a top view of the breast pump adapter of FIGS. 4A-4C.

In this embodiment, a central ring 112 is optionally included. As noted above, the first and second ends 104, 106 of the adapter 100 can each optionally include one or more tabs 108, as shown in FIGS. 2 and 3, which can be used for stretching the respective end when coupling or decoupling the end to or from a bottle coupling 15 or opening of the storage container 20. In other examples, such as the example shown in FIGS. 4A-4D, an adapter 200 need not include tabs or the like.

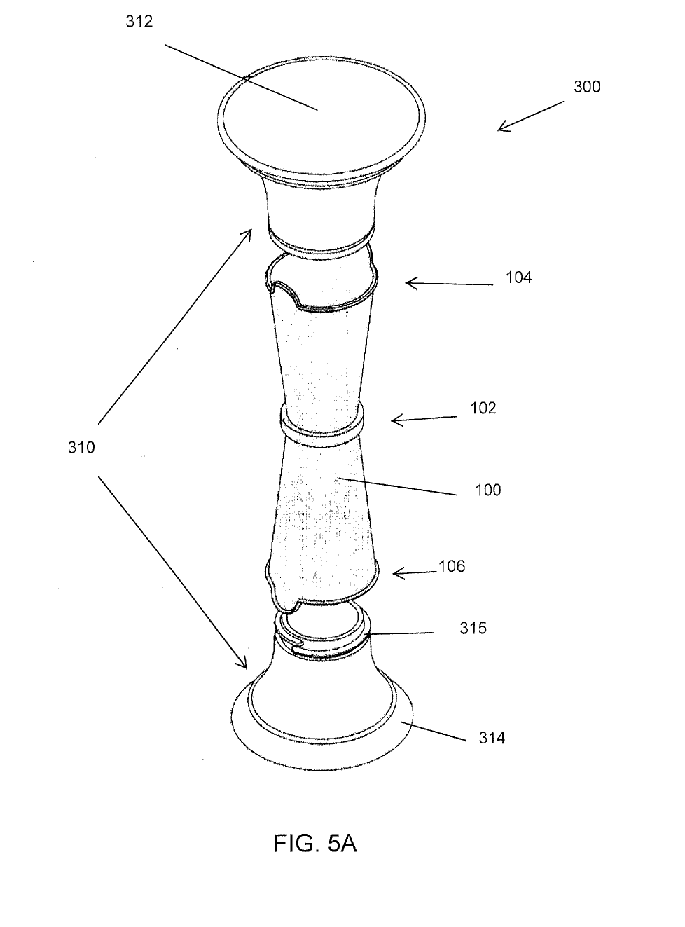

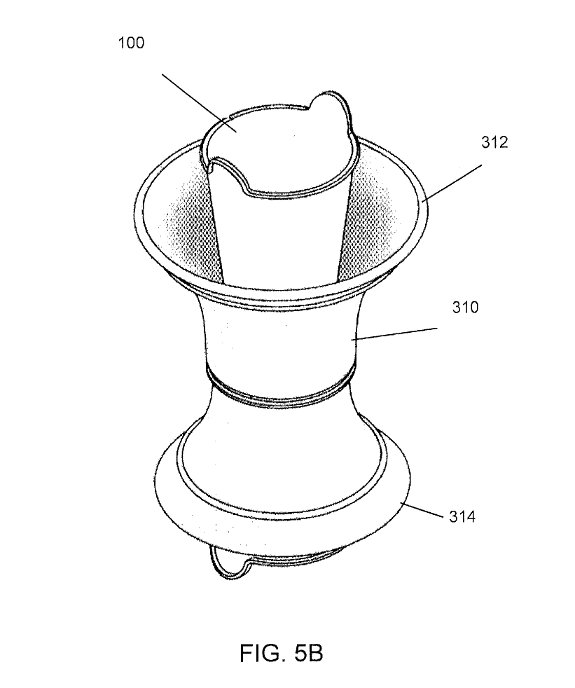

In various embodiments, the breast pump adapter 100 of FIGS. 1A-3 or adapter 200 of FIGS. 4A-4D may include an outer housing 310, for example, as shown in the embodiment of FIGS. 5-9.

The housing 310 can be a one-piece or multi-piece unit. In various embodiments, the housing 310 is preferably formed of a rigid plastic or other material for providing protection and rigidity to the flexible material (e.g., silicone or the like) of the adapter.

FIG. 5A is a perspective view of an embodiment of a breast pump adapter kit 300 in disassembled form having the outer housing 310 within which the adapter 100 shown above can be inserted. FIG. 5B is a perspective view of the breast pump adapter kit 300 of FIG. 5A in assembled form. The adapter 100 is positioned within the housing 310, the housing 310 preferably surrounds at least the central region 102 of the adapter 100.

In this embodiment, the housing 310 includes a pump-side piece 312 structured to surround a portion of the first end 104 of the adapter 100. The housing 310 includes a bottle-side piece 314 structured to surround a portion of the second end 106 of the adapter 100. Because the adapter 100 is flexible (and pliable) it can be compressed to receive the surrounding housing pieces 312, 314, which have a horizontal joint in this embodiment. In other embodiments, the two pieces 312, 314 of the housing 310 could have a vertical joint or other joint.

The two pieces 312, 314 of the housing 310 can be made to mate together, in a secure but removable manner, to create the fully assembled adapter kit 300 of FIG. 5B. Various mechanisms can be used to join the pump-side piece 312 with the bottle side piece 314, e.g., a snap fitting or a screw-type threaded fitting. In this embodiment, a screw-type threaded fitting 315 is used, but not limited thereto. Here, the pump-side piece 312 has a female (or internal) threading (not shown) and the bottle-side piece 314 has a corresponding male (external) threading 315, which cooperatively form a central joint.

In one-piece or two-piece embodiments, the housing 310 can be made of a block or mold of a substantially rigid material, e.g., hard plastic. In such a case, the pliable adapter can be compressed and inserted into the housing 310.

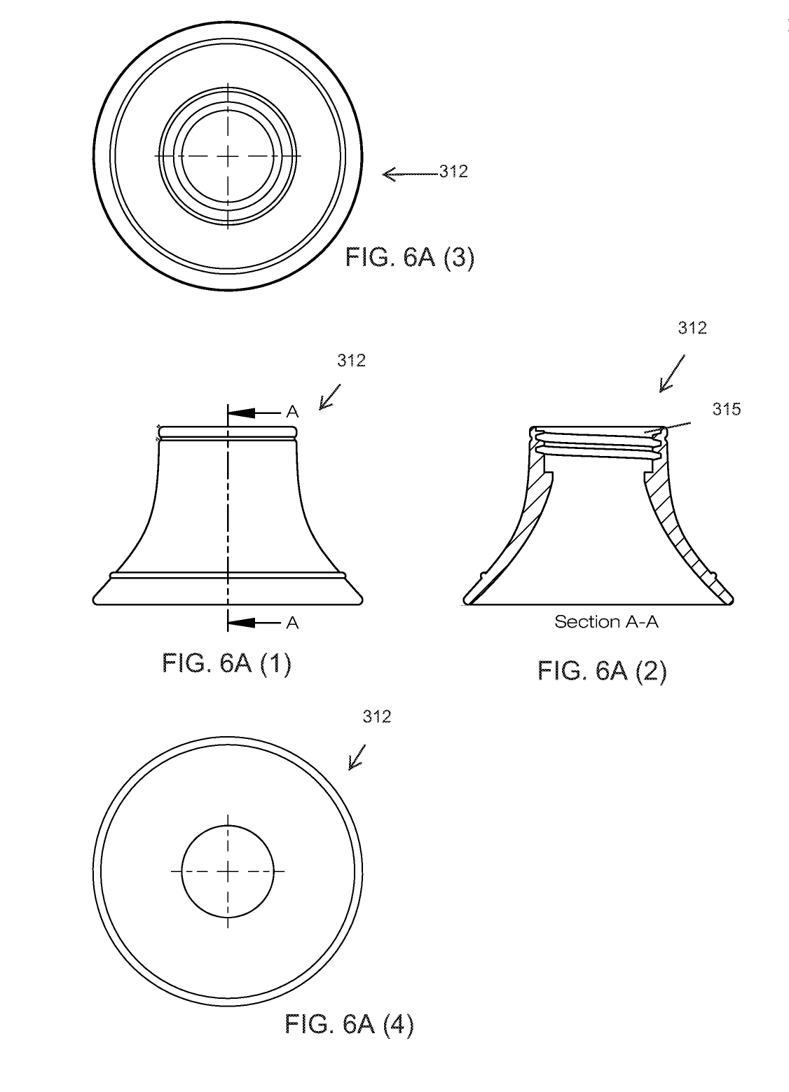

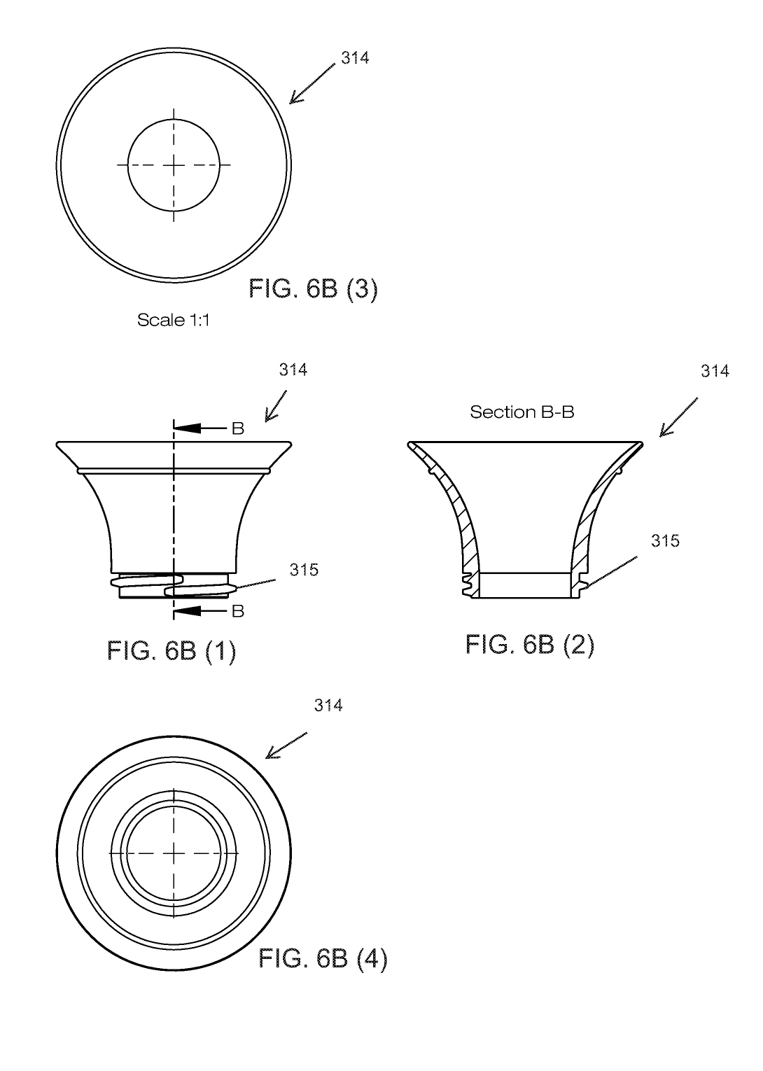

FIG. 6A includes various views of the pump-side piece 312 of the housing 310 of FIGS. 5A and 5B. FIG. 6A(1) is a front view of the pump-side piece 312. FIG. 6A(2) is a cross-sectional view of the pump-side piece 312 of FIG. 6A(1), taken along line A-A of FIG. 6A(1). FIG. 6A(3) is a bottom view and FIG. 6A(4) is a top view of the pump-side piece 312 of the housing of FIGS. 5A and 5B. The top end of the pump-side piece 312 can be fluted or flared so that it does not substantially interfere with stretching the first end 104 of the adapter 100.

FIG. 6B include various views of the bottle side piece 314 of the housing 310 of FIGS. 5A and 5B. FIG. 6B(1) is a front view of the bottle-side piece 314. FIG. 6B(2) is a cross-sectional view of the bottle-side piece 314 of FIG. 6B(1), taken along line B-B of FIG. 6B(1). FIG. 6B(3) is a bottom view and FIG. 6B(4) is a top view of the bottle-side piece 314 of the housing 310 of FIGS. 5A and 5B. The bottom end of the bottle-side piece 314 can be fluted or flared so that it does not substantially interfere with stretching the second end 106 of the adapter 100.

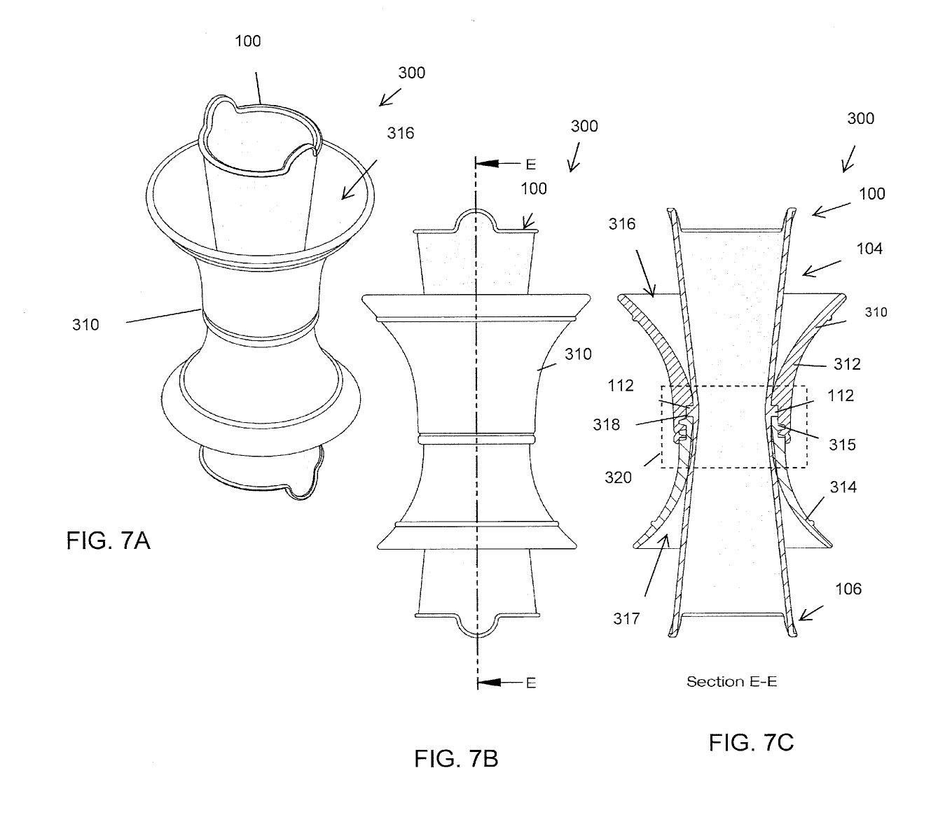

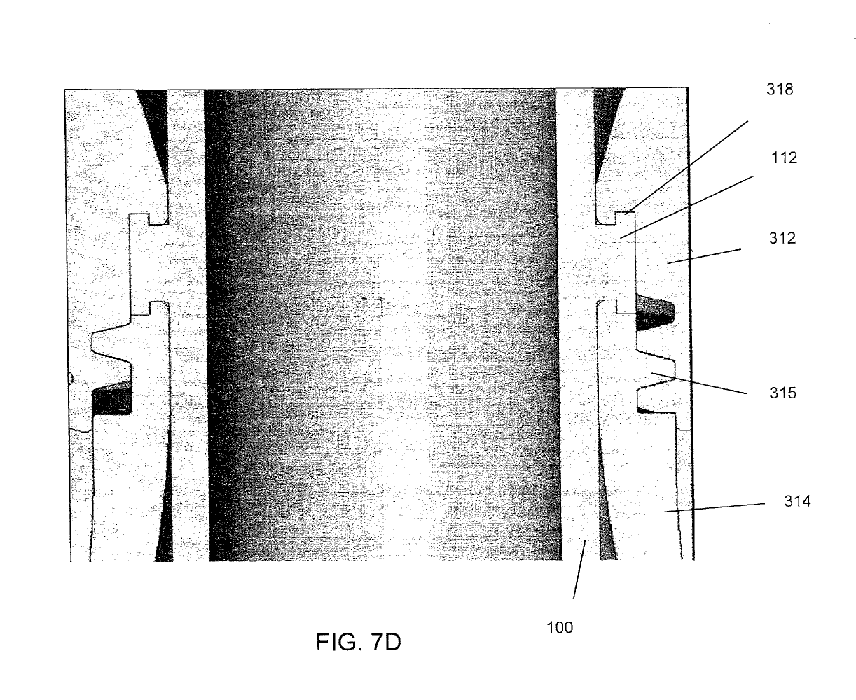

FIG. 7A is a perspective view of the adapter kit 300 of FIG. 5B, with assembled housing 310 and inserted adapter 100. FIG. 7B is a front view of the adapter kit 300 of FIG. 5B, with assembled housing 310 and inserted adapter 100. FIG. 7C is a cross-sectional view of the adapter kit 300 of FIG. 7B taken along lines E-E. FIG. 7C includes a callout showing, in FIG. 7D, a magnified view of details of a joint 320 between the pump-side piece 312 of the housing 310 and the bottle-side piece 314 of the housing 310, for this embodiment. The adapter 100, which has flexible first 104 and second ends 106 in this embodiment, extends through the housing 310. That is, the first end 104 of the adapter 100 protrudes from a first opening 316 formed in a first end of the housing 310 in the pump-side piece 312. And the second end of the adapter protrudes from a second opening 317 formed in a second end of the housing 310 in the bottle-side piece 314. Thus, the flexible first 104 and second 106 ends of the adapter 100 extend from both of the first and second ends of the housing 310 to enable coupling of the adapter ends to the bottle coupling of the breast shield assembly and to the bottle opening of a milk bottle (see FIG. 2).

As shown in FIGS. 7C-D, the adapter 100 can be maintained within the housing 310 by a protruding center ring 112 of the adapter 100 described herein above. In this embodiment, the ring 112 can be securely maintained within, or mate with, a notch, channel, indentation(s), or the like formed within the housing 310, referred to herein as a “channel 318.” In the two-piece housing embodiment, the channel 318 is formed at the interface 320 of the male 312 and female 314 threaded ends 315 of the housing 310, as is shown in the example of FIGS. 7C-D. In this embodiment, the channel 318 includes at least one portion formed by the pump side piece 312 and at least one portion formed by the bottle side piece 314, i.e., at their joint 320. The ring 112 is dimensioned or sized and shaped to fit and be maintained within the channel 318 during use, e.g., has a height and depth that corresponds to the channel 318. Such a channel 318 could also be formed in a single-piece housing. Maintaining the ring 112 within the channel 318 provides further protection against collapsing during use.

In other embodiment, different arrangements could be used, e.g., a plurality of rings and channels, one or more protrusions or projections and one or more corresponding holes formed in the housing 310, and so forth.

The first 312 and second 314 ends of the housing 310 can be flared, similar to, but wider than the unstretched ends of the adapter 100. Preferably, the length of the housing 310 and the flaring of the ends 312, 314 of the housing 310 are such that they do not unnecessarily inhibit the stretching of the first 104 and second 106 ends of the adapter 100 for coupling to the bottle coupling of the shield assembly and the bottle opening of the milk bottle (see FIG. 2). Therefore, the length of the housing 310 can be less than that of the adapter 100, so that the first 104 and second 106 ends of the adapter 100 extend from the first 312 and second 314 ends of the housing 310, respectively. Ultimately, however, the flaring of the housing 310 in this manner can act as a limiter for preventing excessive stretching of the first 104 and second 106 ends of the adapter 100, so that the first 104 and second 106 ends of the adapter 100 cannot be stretched beyond a physical limit imposed by the flared ends 312, 314 of the housing. This may be useful for avoiding damage to the adapter and/or for avoiding an unsafe condition where overstretching of the adapter 100 may cause the adapter 100 to fail during use. Such failure could be or include, as examples, tearing of the adapter 100 or a compromised seal.

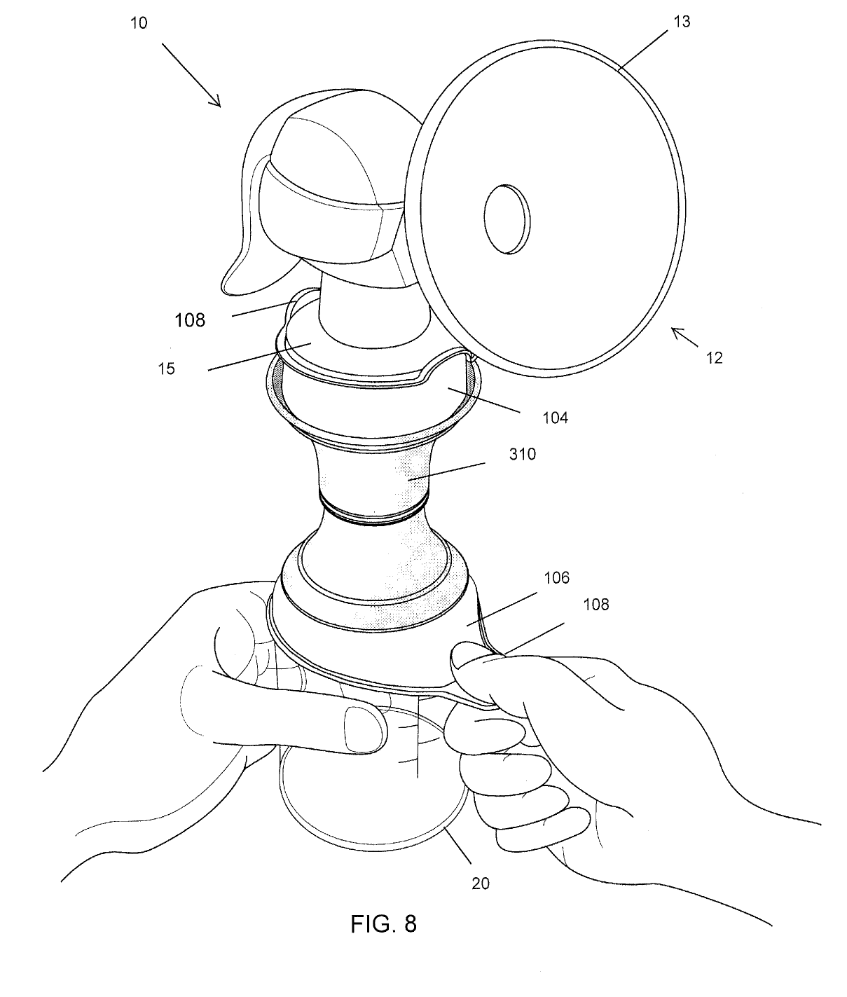

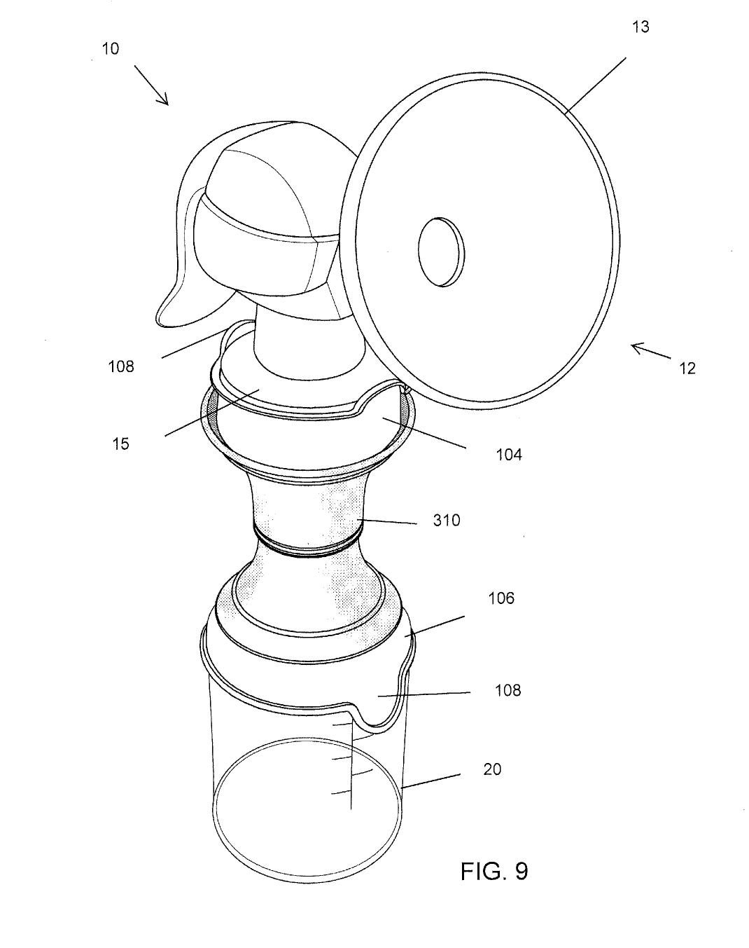

FIGS. 8 and 9 are similar to FIGS. 2 and 3, except in FIGS. 8 and 9 the adapter 100 is positioned within the housing 310. The bottle coupling 15 of the breast shield assembly 12 is typically an internally threaded opening (not shown), i.e., threaded about an interior surface of the bottle coupling 15. An external surface of the bottle coupling 15 can be smooth or textured. The first end of the adapter 104 is stretched about the exterior surface of the bottle coupling 15 to enclose and seal the opening with an airtight seal (see FIGS. 8 and 9). A tab 108 on the first end 104 of the adapter 100 is pulled to facilitate stretching of the first end 104 about the bottle coupling 15. FIGS. 8 and 9 show the first end in place for use.

The opening of the container 20, referred to as a bottle opening, is typically an externally threaded opening, i.e., the thread is at an outer surface of the container 20. The threading of the bottle opening is formed to be compatible with the internal threading of a specific breast shield assembly bottle coupling 15. The second end 106 of the adapter 100 is stretched about the externally threaded region of the bottle opening to enclose and seal the opening with an airtight seal. A tab 108 on the second end 106 of the adapter 100 is pulled to facilitate stretching of the second end 106 about the bottle opening. FIG. 8 shows the stretching and FIGS. 8 and 9 show the second end 106 in place for use.

The foregoing embodiments illustrate an adapter coupled between a breast pump and a container, but are not limited thereto. For example, in other embodiments, an adapter (without or without a housing) may be used to couple a different fluid source system, in place of a breast pump, with different bottles. The figures demonstrate the universal nature of the adapter, fitting different size bottles, effectively achieving a secure, airtight seal and path between the bottle and fluid source. For example, the fluid source may operate in a similar manner as a breast pump.

FIGS. 10-12 show different embodiments of an adapter, each having a second end that is flexible and a first end that is rigid and threaded. In FIG. 10, the adapter 400 has a first end 404 that is externally threaded and configured to fit a specifically sized bottle coupling of a breast pump shield assembly, but a second end 406 that is flexible to be used with a variety of different sized bottle openings, as described above.

In FIG. 11, the embodiment is a two-piece embodiment where the first end 504 of an adapter 500 includes a flexible portion that is constructed for insertion into a rigid (e.g., plastic) cap having a hole in its center for accommodating insertion of the flexible portion, which includes the flexible second end 506 of the adapter.

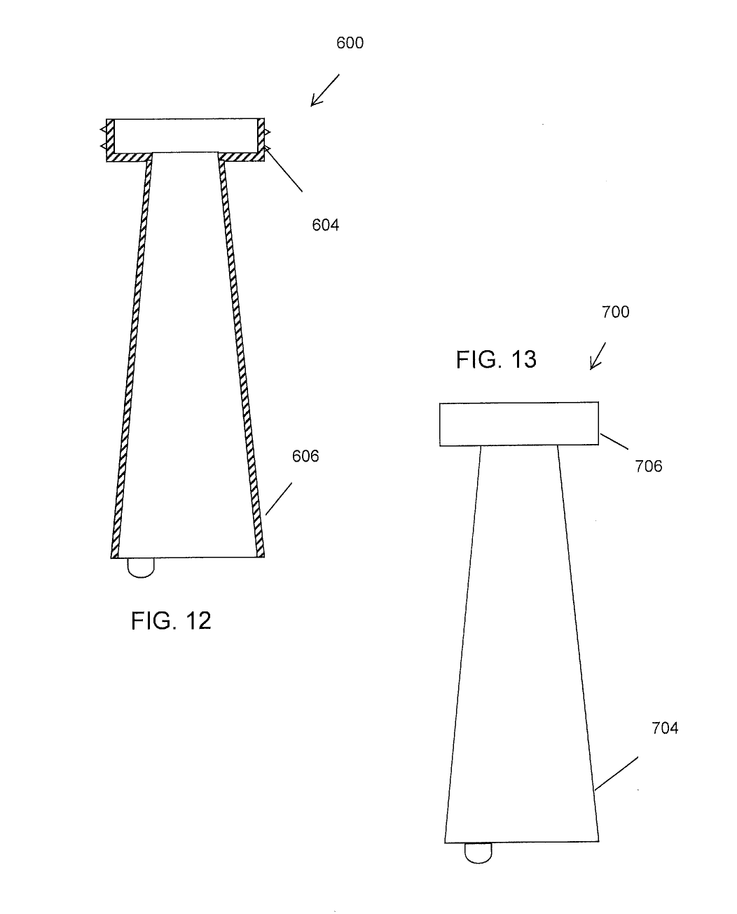

In FIG. 12, the embodiment is a one-piece embodiment where the first end 604 of an adapter 600 is sufficiently rigid to screw into a bottle coupling of a shield assembly and the second end 606 is flexible to fit over a bottle opening of no particular size.

FIGS. 13-15 show different embodiments of an adapter having a first end that is flexible and a second end that is rigid and threaded. In FIG. 13, an adapter 700 has a second end 706 that is internally threaded (not shown) and configured to fit a specifically sized bottle opening, but the first end 704 is flexible to be used with a variety of different bottle couplings of a breast pump shield assembly, as described above.

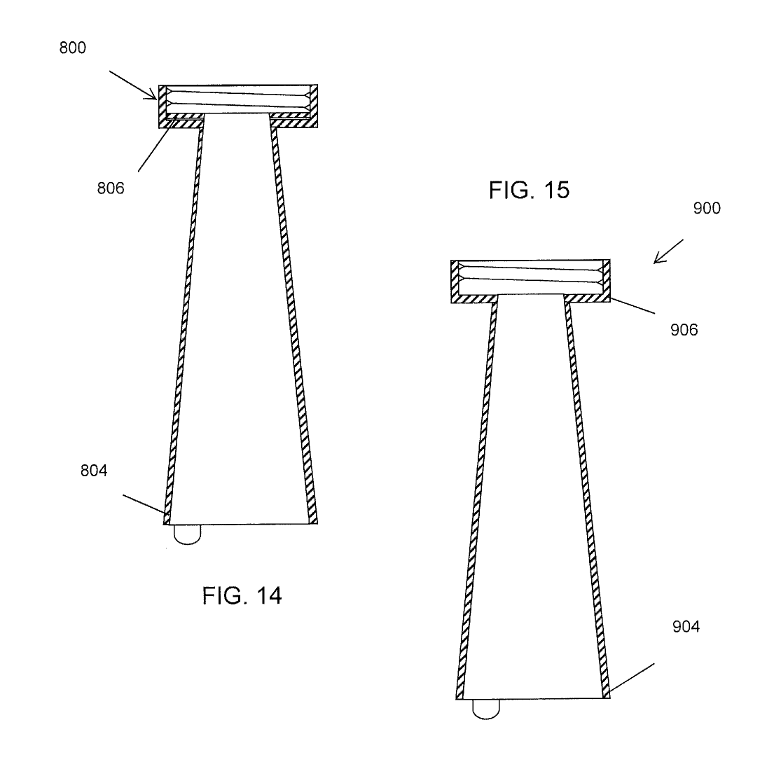

In FIG. 14, the embodiment is a two-piece embodiment where at the second end 806 of an adapter 800 a flexible portion is inserted into a rigid (e.g., plastic) cap having a hole in its center for accommodating insertion of the flexible portion, which includes the flexible first end 804 of the adapter.

In FIG. 15, the embodiment is a one-piece embodiment where the second end 906 of an adapter 900 is sufficiently rigid to screw onto a bottle opening and the first end 904 is flexible to fit over a bottle coupling of a breast pump shield assembly of no particular size.

FIG. 16A is a front view of another embodiment of a breast pump adapter 1000, in accordance with aspects of the inventive concept. FIG. 16B is a top view of the breast pump adapter 1000 of FIG. 16A. FIG. 16C is a cross-sectional view of the breast pump adapter 1000 of FIGS. 16A and 16B. FIG. 16D is a perspective view of the breast pump adapter 1000 of FIGS. 16A-16C. FIG. 16E is a perspective view of the breast pump adapter 1000 of FIG. 16A having a bottle end stretched for coupling to a bottle opening. FIG. 16F is a perspective view of the breast pump adapter 1000 of FIG. 16E coupled to the bottle opening. FIG. 16G is a perspective view of the breast pump adapter 1000 of FIG. 16A having a breast pump end stretched for coupling 15 to a breast shield assembly. FIG. 16H is a perspective view of the breast pump adapter of FIG. 16G coupled to the breast shield assembly. And FIG. 16I is a perspective view of the breast pump adapter of FIGS. 16A, F, and H coupled to both a bottle 20 and a breast shield assembly coupling 15.

In this embodiment, the breast pump adapter 1000 takes a hollow, generally tubular form. The adapter 1000 is configured to create an enclosed, airtight fluid path between a shield assembly coupling 15 of a breast pump, or related device for receiving fluid from a human breast, and a bottle 20 or related storage container at the end of the fluid path. The adapter 1000 includes a central region 1002, a first end 1004, and a second end 1006. The portion of the adapter 1009 between the first end 1004 and second end 1006 may be referred to as an intermediate region, inclusive of the central region 1002.

The first end 1004 and second end 1006 of the adapter 1009 can each include one or more tabs 1008, or lips or other gripping structures, similar to those described in other embodiments herein. Additional details on the tabs 1008 are therefore not repeated for brevity.

The first end 1004, the second end 1006, and intermediate region 1002 can be formed of the same material, or different materials. In some embodiments, the entire adapter 1000 can be flexible, or alternatively, the intermediate region or central region 1006 can be rigid while one or both of the first 1004 and second 1006 ends are flexible. Portions of the adapter may include protrusions, bumps, or the like for gripping, as shown. In some embodiments, the protrusions extend from a portion of the adapter wall at the first end 1004 and the second end 1006. Although the protrusions are shown on the adapter 1000 of FIGS. 16A-16I, similar or same protrusions may equally apply in other embodiments, for example, adapter 100 illustrated and described herein.

The seal formed by at least one of the first end 1004 or the second end 1006 of the adapter 1000 is a conformal air tight seal that maintains the suction or vacuum of a breast pump, whether electric or manual. For example, the coupling 15 shown in FIGS. 16G-16I may be part of the breast pump. The depicted embodiments show two flexible (adaptable) ends 1004, 1006. However, the ends can have configurations, shapes, flexibility or rigidity, and/or other features or characteristics similar to or the same as those of other embodiments illustrated and described with respect to FIGS. 1-15.

In some embodiments, as shown in FIG. 16C, a portion 1003 of a wall of the adapter 1000 at the central region 1002 has a thickness and/or density that is greater than the other regions of the adapter 1000. This portion 1003 may be functionally similar to the support portion 112 illustrated and described in other embodiments with respect to providing stability and rigidity to the adapter 1000 e.g., t prevent collapsing of the adapter 1000 when a vacuum is applied from the breast pump. The thickening of the walls of the adapter in this manner could be provided in combination with protrusions, projections, cavities, depression, or combinations thereof.

In some embodiments, the central portion 1003 can be formed of a different material than other portions of the adapter 1000, i.e., different from the first end 1004 and/or second end 1006. In some embodiments, an additional material may be added in the intermediate region to provide added rigidity, thickness, density, or other desired features.

In the embodiment of FIGS. 16A-I, the first and second ends 1004, 1006 are flared but the intermediate region 1002 is thickest in the central region 1112 and then the adapter gradually widens from the central portion 1003 toward each of the first and second ends. In that regard, central portion 1003 can be considered similar to ring 112 discussed above.

While the foregoing has described what are considered to be the best mode and/or other preferred embodiments, it is understood that various modifications can be made therein and that the invention or inventions may be implemented in various forms and embodiments, and that they may be applied in numerous applications, only some of which have been described herein.

It is intended by the following claims to claim that which is literally described and all equivalents thereto, including all modifications and variations that fall within the scope of each claim.

What is claimed is:

1. A breast pump adapter, comprising:
an elongated body having a first end, a second end, and an intermediate region, wherein:
the first end is formed of a flexible material that is configured to stretch around a breast shield assembly bottle coupling to form an airtight seal; and/or
the second end is formed of a flexible material that is configured to stretch around a storage container opening to form an airtight seal.

2. The breast pump adapter of claim 1, wherein the first end is formed of the flexible material configured to stretch around a breast shield assembly bottle coupling to form the airtight seal.

3. The breast pump adapter of claim 2, wherein the second end is threaded to mate with threads of the breast shield assembly bottle coupling.

4. The breast pump adapter of claim 1, wherein the second end is formed of the flexible material configured to stretch around a storage container opening to form the airtight seal.

5. The breast pump adapter of claim 4, wherein the first end is threaded to mate with threads of the storage container.

6. The breast pump adapter of claim 1, wherein both the first end and the second end are formed of the flexible material and configured to form the respective airtight seals.

7. The breast pump adapter of claim 1, wherein the storage container is a milk bottle.

8. The breast pump adapter of claim 1, wherein the adapter is a single piece formed of one material.

9. The breast pump adapter of claim 1, wherein the material is or includes at least one of silicone and rubber.

10. The breast pump adapter of claim 1, wherein at least one of the first end or second end is flared and has an opening that is wider than a width of an interior of the adapter at the intermediate region.

11. The breast pump adapter of claim 10, wherein the first and second ends are both flared.

12. The breast pump adapter of claim 1, wherein at least one of the first and second ends includes a pull tab that facilitates stretching of the corresponding first end and/or second end.

13. The breast pump adapter of claim 1, wherein the intermediate region includes a substantially rigid region that resists collapsing under a vacuum pressure applied via the breast shield assembly by a breast pump.

14. The breast pump adapter of claim 13, wherein the substantially rigid region includes a ring formed about a central region of the adapter.

15. The breast pump adapter of claim 1, further comprising:
a rigid housing configured to receive, surround, and secure therein a portion of the adapter, wherein a length of the housing is less than a length of the adapter and the first end of the adapter extends beyond a first end of the housing and a second end of the adapter extends beyond a second end of the housing.

16. The breast pump adapter of claim 15, wherein the adapter includes a protrusion in the intermediate region and the housing includes an intermediate channel configured to receive the corresponding protrusion of the adapter to secure the adapter within the housing.

17. The breast pump adapter of claim 1, wherein the adapter comprises a wall that extends from the first end to the second end, and wherein a portion of the wall at the intermediate region has a thickness that is greater than a thickness of portions of the wall at the first and second ends, respectively.

18. A breast pump system, comprising:
a breast pump having a breast shield, a pump valve, and a bottle coupling;
a breast pump adapter that can be coupled between the breast pump bottle coupling and a storage container opening to form an airtight fluid path between the breast pump bottle coupling, the breast pump adapter, and the storage container,
the breast pump adapter comprising an elongated body having a first end and a second end and an intermediate region, wherein:
the first end is configured to couple to the breast pump bottle coupling to form an airtight seal; and
the second end is formed of a flexible material that is configured to stretch around a storage container opening to form an airtight seal.

19. The breast pump system of claim 18, wherein:
the first end is formed of the flexible material that is configured to stretch around the breast pump bottle coupling to form the airtight seal; and
the intermediate region includes a substantially rigid region that resists collapsing under a vacuum pressure applied via the breast shield by a breast pump.

20. A breast pump adapter, comprising:
an elongated body having a first end, a second end, and an intermediate region, wherein:
the first end is formed of a flexible material that is configured to stretch around a breast shield assembly bottle coupling to form an airtight seal;
the second end is formed of a flexible material that is configured to stretch around a storage container opening to form an airtight seal; and
the intermediate region includes a substantially rigid region that resists collapsing under a vacuum pressure applied via the breast shield assembly by a breast pump.
